(19) United States
(12) Patent Application Publication
(10) Pub. No.: US 2006/0154197 A1
(43) Pub. Date: Jul. 13, 2006

(54) Anatomical mini-illuminator for lingual cavity

(76) Inventor: Vincenzo Gargiulo, Roma (IT)

Correspondence Address: YOUNG & THOMPSON, 745 SOUTH 23RD STREET, 2ND FLOOR, ARLINGTON, VA, 22202, US

(21) Appl. No.: 10/562,834

(22) PCT Filed: Jul. 1, 2004
(86) PCT No.: PCT/IT04/00383

§ 371 Date: Dec. 30, 2005

(30) Foreign Application Priority Data
Jul. 2, 2003 (IT) RI 2003 A 00005

(51) Int. Cl. A61C 1/00; A61C 5/00; A61F 5/00

(52) U.S. Cl. 433/29; 433/140; 606/242

(57) ABSTRACT

Apparatus for the illumination of the vestibular/lingual cavity, having a symmetric couple of retractors for the oral cavity. A set of point-like light sources are distributed on each retractor, the light sources corresponding to respective terminations of an optical fibre which is inclined by a specific angle with respect to the ideal plane of the closed set of teeth. It is possible to obtain a complete, uniform, and close illumination of the lingual and vestibular sides of each tooth. The couple of retractors is arranged on a support structure, which forms: the inlet element for the lower extremity of the optical fibres, the regulation device used for adjusting the initial (normal) aperture of the retractor.

### TECHNICAL FIELD OF THE INVENTION

The invention generally relates to instrumentation used for dental inspection, and more particularly it concerns an oral retractor, supporting an illumination system for uniformly illuminating the oral cavity, to be arranged in the forward vestibule of mouth, and wherein said illumination system is integral to the structure of the retractor itself.

### BACKGROUND ART

In dentist's surgeries illumination units are largely used which have an illumination device at one end of a movable arm, so as to direct the light from an oblique upper position, towards the chair on which the patient is lying.

The plane of reflection of the illumination device is curved, so that the light is directed inside the mouth during the dental diagnosis or during the treatment.

The lamp—or light source—of the illumination device is located within the surface of the curved reflection plane, the aim—which is difficult to attain in a satisfactory manner—being to minimise the regions of shadow within the mouth even when the dentist intercepts (cuts off) part of the light beam or the illumination device.

Obviously, a series of problems result from the use of such illumination systems: repeated adjustments in consequence of the patient's movements and the difficulty in the visualisation of a lateral problem or, in any case, the difficulty deriving from the fact that the operator may interfere with the path extending from the light source to the mouth.

On the other hand, the precision required when one operates on the teeth as well as the poor visibility of their anatomic position, have urged to look for various different solutions in order to provide an acceptable illumination of the operation field. Thus, instrumentation with small optical fibres, scialitic double lamp techniques, light sources located on the operator's head, and various other alternative solutions have been devised to solve this problem, all relying on external light sources directed towards the mouth cavity. This kind of illumination becomes insufficient or even useless when the patient turns round, or when he/she is urged to move in order to facilitate the operator's work.

The position of the scialitic lamp, located behind the operator, really does not permit a direct visibility on the operation field, because the operator is not allowed to intercept (cut off) the path extending from the light source to the mouth. Also, the scialitic lamp is a light source that diffuses an annoying amount of heat onto the operator's shoulders, and it is impossible to always get the maximum benefit out of it because of the hardening of photopolymers, if any, and above all, it must be positioned at a distance of about 80 cm to obtain the maximum concentration of the light (about 22000 Lux), thereby producing sometimes a dazzling effect on the patient. The fact that the scialitic lamp must always be directed towards the relevant zone implies that the handle of the lamp will be a particularly infected region of this electro-medical equipment.

The mini-illuminator according to the present invention aims at solving, with a single apparatus, the above mentioned problems inherent in the light source, the positioning, the opening-out of the patient's mouth, the polymerisation, the highlighting (visualisation) of the dental plaque, and the endoscopic examination. The concept of illumination should be completely changed. The light should be present only in the operation field and the relevant zone should be totally free (clear), due to the respective retractor's action.

The object of the present invention is therefore to provide an illumination system which is no more positioned outside, and moreover, which is no more subject to repeated adjustments due to the patient's movements, the necessity of visualising a lateral problem, or simply due to that fact that the operator assumes a position between the light source and the mouth; instead, the apparatus will be positioned inside the oral cavity and will therefore allow an exact positioning of the light source used during the operation.

A further object of the present invention is to provide a mini-illuminator allowing a direct vision of the operation field without resorting to an alternative light source. Such direct sight of the oral cavity, in a perfectly illuminated field, will not produce any dazzling effect on the patient.

A last object of the present invention is to provide an illumination system of the vestibular/lingual cavity that employs structural elements, materials and technologies which are of a standard kind in the field of the instrumentation used for dental inspection, thereby limiting the cost of the device and facilitating its maintenance. These and other objects resulting from the description given below, are attained by means of a micro-illuminator of the vestibular/lingual cavity, comprising a symmetric couple of retractors (A, B) of the oral cavity. On each retractor there is a set of point-like (punctiform) light sources, each of which derives from the termination—on the external surface of the retractor—of an optical fibre which is tilted according to a specific angle with respect to an ideal plane of the closed set of teeth. In this manner it is possible to obtain a complete, uniform, and close illumination of the vestibular and lingual side of each tooth. Said couple of retractors is arranged on a support structure having an Y-configuration, this structure forming at the same time:

i—the inlet element for the passage of optical fibres through the lower end, said optical fibres branching off on the first and second retractor, and

ii—the regulation device, used for adjusting the initial (normal) aperture or opening of the retractor, the adjustment being performed by coupling the retractor to a semirigid small bow selected from an available set according to the typology of oral cavity on which the operation is carried out and according to the kind of operation.

This apparatus is exactly the opposite of all illumination systems realised so far.

The local mini-illuminator according to the present invention allows the operator, for the first time, to work with a perfect sight, occupying a position directly in front of the patient and without being forced to a compromise between the illumination intensity and the operator's position.

The use of the double retractor allows the orthodontics specialist to completely visualise both dental arches, by perfectly illuminating both the vestibular and the lingual part of the tooth, thereby affording the possibility of using all kinds of orthodontic attachments commercialised nowadays.

The retractor also allows to photograph the dental arches without use of a flash, no shadow regions being formed in this process.

The use of only the right or left semi-arch allows the endodontia specialist or the implant specialist to work also on the octaves, exploiting a light that is emitted from the operation field itself.

The mini-illuminator is suited to emit white light whose intensity may vary in a wide range of values simply by adjusting a respective potentiometer. This light may be converted into yellow light, with the same range of intensities, for allowing a direct vision of the dental plaque. The mini-illuminator is also utilised as an endoscopic lamp, emitting a point-like light beam, and may even be used as a curing (polymerising) lamp in order to harden specific composite materials, by providing the apparatus with a stopwatch specifically designed for the regulation of the hardening of the composite material. The opening-out action performed by the illuminator itself insures a wide and free sight on all the operation zone, thereby permitting to use microscopes or other electro-medical machines that are employed in dentistry and other sectors.

### BRIEF DESCRIPTION OF DRAWINGS

Only for illustrative purposes and without intending to limit the generality and the possible applications, a preferred embodiment of the present invention will next be described with reference to the annexed drawings, in which:

FIG. 1 shows various perspective views of the invention, taken from different directions;

FIG. 2 shows several axonometric views of a mini-illuminator, also shown in FIG. 1, with specific reference to the distribution (arrangement) of the optical fibres on the retractor;

FIG. 3 is a representation of a fitting (accessory) for the mini-illuminator/retractor shown in FIGS. 1 and 2;

FIG. 4 is a representation of several fittings of the mini-illuminator/retractor of FIGS. 1 and 2

FIG. 5 schematically shows a particular condition during an operation.

### DISCLOSURE OF PREFERRED EMBODIMENTS

It must be stated beforehand that for simplicity the views are only schematic and that some specific structural features have been intentionally omitted because their presence is taken for granted and because they are not considered relevant for the description of the preferred embodiment; moreover, in the axonometric and perspective views the same number always corresponds to the same constructive part.

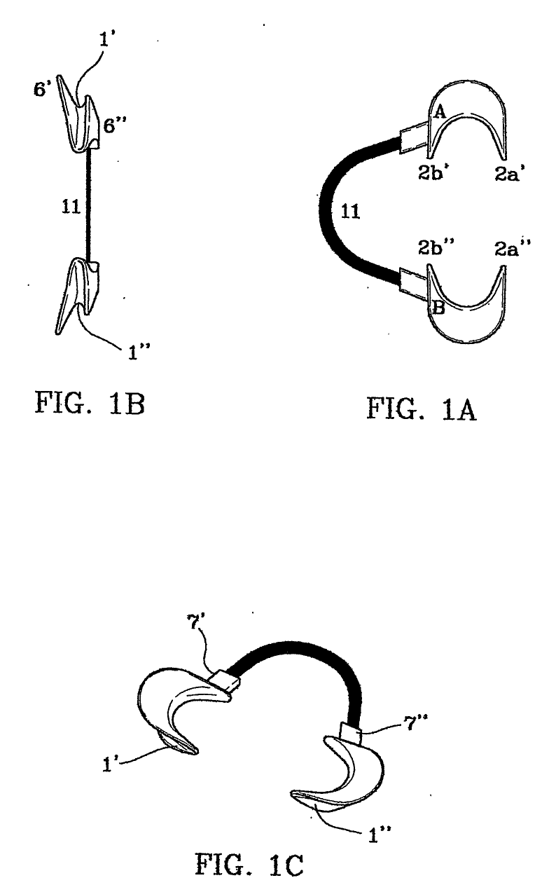

Referring to FIG. 1, it shows several axonometric views of the illumination device according to the invention. Immediately visible are the two retractors A and B, which have an arched form and are provided with respective channels or recesses 1 on their curved external profile, apt to receive the lateral regions of the patient's mouth. From FIG. 1b it may also be clearly seen that in order to obtain a light structure, the two lanes 6′ and 6″ of each retractor, which bound the channel 1, are configured symmetrically. In particular, the first one, 6′, is wider and thicker—this corresponds to the section to be inserted behind the cheek—whilst the second, 6″, is thinner and less cumbersome, and it corresponds to that part of the retractor designed to remain outside the mouth.

Further, it can be noted that the retractor has a rectilinear profile 2 both in its upper and lower extensions. In practice, the improved construction of the mould, that was studied under an anatomical point of view, starts from the horizontal line of the upper lip and ends in the horizontal part of the lower lip, so as to perfectly opening out the mouth, without requiring the use of small mirrors—or the like—which open wide the mouth in a point-like manner and are annoying for the patient.

In other words, the concavity formed by the channel 1 follows (matches) as much as possible the profile of the mouth of the patient in the opened condition of the mouth.

Actually, the mouth, when it is opened wide, laterally forms a circular line which little by little becomes rectilinear when considering the upper or lower part of the lip. The respective anatomic configuration of the retractor, which terminates—at its ends—according to two parallel lines 2a′, 2a″, 2b′ and 2b″, has the effect that the stress points, during the action of opening out, are distributed as much as possible on the maximum extension of the mouth. With this solution it is possible to achieve stable working conditions and the maximum comfort for the patient, whose lips are perfectly and uninterruptedly in contact with the retractor device. This is achieved thanks to the complete absence of mechanical joints and projections of any kind, and particularly because of a uniform application (distribution) of the tension on a load-bearing profile which is made wider.

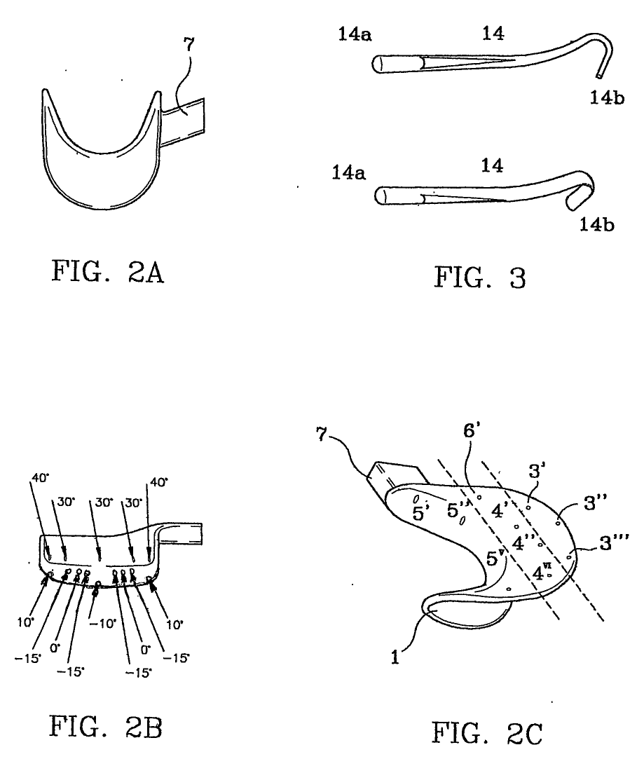

FIG. 2 highlights the presence of “light points” on the retractor. During the moulding process of the retractor, a plurality of inner channels have been provided, each of which receives a respective optical fibre; the latter ends with a specific orientation linked to the particular direction conferred to the optical fibre by the respective through channel. The position of each fibre has been designed (selected) in such a way as to eliminate any region of shadow. In fact, the fibres have different tilts—e.g. 100°-50°-20°—with respect to an ideal plane taken when the set of teeth is in the closed condition. These fibres are arranged in an equidistant manner, both from the inside to the outside, and from top to bottom, taking account of the requirement of a good sight on the operation field, when the set of teeth is in the closed condition and—particularly—when it is in the opened condition.

In the latter condition the operator is able to see the operation point even while he/she is introducing specific instruments (into the mouth), because the positions of the above described optical fibres allow to emit light in addition to the light intercepted by the operator.

The FIGS. 2b and 2c provide more details for a particular and preferred embodiment, by illustrating the direction and the arrangement of optical fibres on the inner side 6′ of the retractor.

In these figures one can see a first series 3′, 3″, 3′″ of three exiting (outgoing) optical fibres located on the projecting—and more external portion—of the retractor, this series being present almost at the very edge of the channel 1. From this position it is possible, in particular, to effectively illuminate the bottom of the lingual/vestibular cavity, and specifically the molar teeth. With regard to the angular plane determined in the closed condition of the set of teeth, the selected angles (tilts) respectively have the values 10°, −10°, and 10°.

A further set of exiting (or outgoing) optical fibres is formed by the six points 4′, 4″, . . . , 4^{IV} arranged in the intermediate zone of the inner retractor's plane and used to illuminate the premolars. The inclination for these light points is respectively equal to: −15°, 0°, −15°, 15°, 0°, −15°.

Finally, a series of five light points 5′, 5″, . . . , 5^{V} is located almost at the inner surface of the projecting structure of the retractor, to permit the sight of the incisive teeth and median lines. For this set of light points preferred angles are respectively 40°, 30°, 30°, 30° and 40°, since they allow to see the lower and upper zone of the front portion of the oral cavity.

In positions located below, the two retractors A and B have respective supports denoted by 7′ and 7″, which project as integral parts of the lower zone of the curvilinear bow-like structure.

Each of these supports 7′ and 7″ is formed like an element having two specific functions:

i—to provide a space for introducing and fitting a small bow 11 of semirigid material, whose opening allows to open wide the mouth of the patient; according to the mouth's size one may use a type of small bow which allows a different degree of opening;

ii—to permit the passage of the optical fibres, whose extremities form the illumination system associated with the retractor.

It should be noted that the arrangement of both supports in the structure of the mini-illuminator—giving rise to an Y-configuration after insertion of the small bow 11-, will naturally occupy a position in the lower zone of the patient's face, where, usually, the lower lip and/or the chin are slightly offset backwards with respect to the lower lip. This solution allows to have a further advantage deriving from the possibility of having a stationary point on which the operator may lay his hand, for instance when he/she is using high-speed instruments.

A series of fittings forming the equipment of the mini-illuminator form a set of devices adapted to be used by any specialist in the field.

The so-called “pen” 14 used to stretch out the cheek and open out the mouth, allows the assistant to totally open out the relevant zone and to illuminate it, using only one hand and—at the same time—shifting the cheek out of the way.

This pen 14 used to stretch out the cheek is shown in FIG. 3 and in FIG. 5 (in this case it has the function to open wide the mouth only on one side to optimise the sight on the vestibule side). This pen forms an instrument provided with a handle 14a and having—at the other end 14b—a curvilinear shape that is bent backward, where the pen has a flattened cross-section in order that the inner surface of the bent portion may perfectly fit together with the retractor's surface—that is to say, the arcuated profile (curvature) of the section of the pen that is bent backwards adapts to the retractor's profile. In this manner it is possible to effectively displace the retractor, e.g. laterally, towards the outside, downwards, upwards, or backwards.

This displacement of the retractor, performed by means of the pen 14 for stretching the cheek, will be very useful for the operator who can operate on the tooth from a larger distance and feel more free while operating.

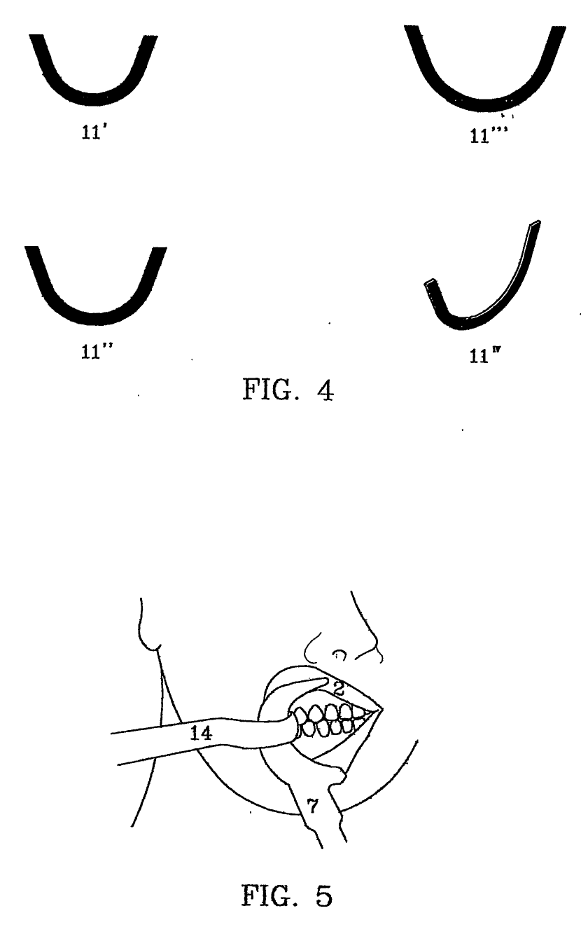

FIG. 4 shows a set of small bows 11 whose function has already been explained.

This set features different apertures, so that each bow can fit to a particular kind of oral cavity. Small bows will also be available which have not the same proportions on their two opposite sides, like—for instance—the type 11^{IV}, these small bows being used in operations in which the oral cavity must be opened out with a specific, asymmetric bias, acting only on one side of the patient's mouth.

In conclusion, the availability of four different types of “small bows” 11′, 11″, . . . 11^{iv} allows a perfect opening out of the patient's lips, without being invasive. An endoscopic lamp, realised by means of a plastic fibre with a surface area of 6 square millimetres and having a power of 150 W, allows optimisation of the visualisation of inter-proximal caries, and if it is positioned behind the tooth, it permits the visualisation of the tooth through the transmitted light, giving the possibility of diagnosing micro-fractures of the tooth or of identifying implants contained therein. The equipment is completed by a polymerising lamp which is realised using a cold light emitting diode with a wavelength of 475/525 nanometres.

In a variant of the preferred solution the mini-illuminator is arranged on the supporting frame of the sheath normally used to obtain a “dam-like effect” on the teeth to be operated on.

In fact, as is well known, in case only one tooth or only a reduced number of teeth need to be treated, some dentists employ a technique that isolates the respective tooth by means of a perforated sheath which creates a passage for this particular tooth. In practice, due to the tension applied on the sheath, the latter is put (fitted) around the tooth so as to isolate it from undesired agents, such as the saliva.

Thus, an obvious variant of the above described system including the mini-illuminator located inside the retractor, consists in arranging the optical fibres on the frame used to stretch the dam, which frame normally forms a metallic U-shaped element; also in this embodiment one obtains an optimum localised illumination.

### ADVANTAGES AND INDUSTRIAL APPLICABILITY OF THE INVENTION

Summing up, the present invention is based on two advantageous and innovative concepts:

the first concept consists in emitting light from the inside to the outside and not vice versa, in contrast to the methods employed so far;

the second concept resides in allowing a direct sight for any position occupied by the patient.

Moreover, when using the mini-illuminator according to the present invention, the illumination does not vary notwithstanding the movements which the patient is obliged to do.

The retractor in front and between the two dental semi arches is provided in polycarbonate and different sizes of the bows adapt the illuminator to a small, a middle or a great oral cavity.

The light emitted by a small 150 W-lamp and transmitted by an optical fibre cable could alternatively be transmitted by other fibres made of glass or plastics.

The combination of the opening-out and illumination effects could be achieved in a separate manner by employing two fibre bundles or the like. The fibres could also be channelled inside metallic retractors, using a temporary lodging of fibres or equivalent light transmission systems. For what concerns the fittings, in order to obtain a yellow or a blue light, depending on the application, these kinds of light may be transmitted by means of isolated fibre bundles whose ends may be provided with a filter to obtain the desired light colour.

Although the description has not so far described such elements, the presence of energy supply apparatus like electronic regulation circuits, toroidal transformers, at least one lamp and a cooling system, is obviously implied. The typical features of the illuminator are: the delivery of a white light at 150 W, the possibility of adjusting the intensity from 1 Lux to 22000 Lux, a stopwatch operating in the range from 10 to 50, a remote control for turning on the device—for instance a control pedal—, a housing made of insulating plastics.

The optical fibres of the equipment include rapid-connection means in order to be able to separately use yellow, blue, or white light, these rapid-connection means being utilised also in the retractor (provided with optical fibres).

The disclosed illumination system does not imply any risks since light transmission is obtained by means of optical fibres and not by employing electric cables.

Among the most advantageous features there is the fact that all parts in contact with the mouth can subsequently be autoclaved, and further, the biocompatibility of the material used to manufacture the illuminators.

The mini-illuminator according to the present invention is a revolutionary system which totally changes the methods of working in dentistry. It maximises the functionality with regard to the illumination of the operation field, the deftness and sight for the operator, the passive collaboration by the patient, and the assistant's efficiency.

1-10. (canceled)

11. Apparatus for the illumination of the vestibular/lingual cavity, comprising a symmetrical couple of retractors (A,B) for the oral cavity, each retractor having a series of punctiform light sources distributed thereon and deriving each from a corresponding termination of an optical fibre on the external surface of the retractor, the apparatus producing a complete, uniform and close illumination from the inside of the mouth, both from the lingual side and from the vestibular side of each tooth; said couple of retractors being located on a support structure (7, 7′,7″) arranged on an U-like configuration, and said structure (7,7′,7″) providing at the same time an inlet, through its lower termination, of optical fibres that branch off on the first (A) and second (B) retractor; the apparatus comprising also regulation means for the adaptation of the normal or initial opening of the retractor through the introduction in said support structure (7,7′,77″) of a semirigid small-bow, the latter being selected from an available set according to the typology of oral cavity on which the operation is to be performed and according to the kind of operation; said optical fibres which end on the surface (6′) of the retractor having differing tilts, said fibres being however arranged equidistantly, both from the inside to the outside and from top to bottom, taking account of the visualization requirement of the operation field both in the closed and in the opened condition of the set of teeth; the apparatus being characterised in that three zones are selected for the arrangement of the punctiform light sources on the surface (6′) of the retractors:
a first series (3) of outgoing optical fibres being arranged in the projecting and more external part of the retractor, almost at the limit of the edge of the channel (1), to effectively illuminate, in the first place, from this position, the bottom of the lingual/vestibular cavity and in particular the molar teeth;
a further series of outgoing optical fibres corresponding to points (4) located in an intermediate zone of the inner plane of the retractor, said points being suited to illuminate the premolars, and
a further series of light points, or punctiform light sources (5), being arranged approximately at the inner surface of the projecting structure of the retractor, in order to illuminate the incisive teeth and the median lines.

12. Apparatus for the illumination of the vestibular/lingual cavity, comprising a symmetrical couple of retractors (A,B) for the oral cavity, each retractor having a series of punctiform light sources distributed thereon and deriving each from a corresponding termination of an optical fibre on the external surface of the retractor, the apparatus producing a complete, uniform and close illumination from the inside of the mouth, both from the lingual side and from the vestibular side of each tooth; said couple of retractors being located on a support structure (7,7′,7″) arranged on an U-like configuration, and said structure (7,7′,7″) providing at the same time an inlet, through its lower termination, of optical fibres that branch off on the first (A) and second (B) retractor; the apparatus comprising also regulation means for the adaptation of the normal or initial opening of the retractor though the introduction in said support structure (7,7′,7″) of a semirigid small-bow, the latter being selected from an available set according to the typology of oral cavity on which the operation is to be performed and according to the kind of operation; characterised in that a set of small bows (11) is available to the apparatus, each of them being suited to a corresponding type of oral cavity, and to a respective small-size, medium-size, or large-size opening of the mouth on which the operation is to be carried out.

13. Apparatus for the illumination of the vestibular/lingual cavity according to claims 12, wherein some of the available small bows have not the same length and/or do not cause the same opening-out on the two opposite sides thereof, and are employed in operations requiring, in order to open out the oral cavity, a specific asymmetric bias on only one side of the patient's mouth.

14. A method for the illumination of the vestibular/lingual cavity which comprises inserting the apparatus of claim 11 into the vestibular/lingual cavity and using said apparatus as an endoscopic lamp to optimize the visualization of inter-proximal caries and to permit the examination of the tooth through the transmitted light, when it is positioned behind the tooth in order to diagnose micro-fractures of the tooth or implants contained therein.

15. A method according to claim 14, wherein the apparatus is a curing lamp, having a diode which emits cold light to cause hardening of composites, the apparatus being provided with a stopwatch specific for the regulation of the composites' hardening.
